(19) United States
(12) Patent Application Publication
(10) Pub. No.: US 2016/0166679 A1
(43) Pub. Date: Jun. 16, 2016

(54) METHOD OF TREATMENT USING FOLATE CONJUGATES AND TYROSINE KINASE INHIBITORS

(71) Applicant: Purdue Research Foundation, West Lafayette, IN (US)

(72) Inventors: Philip S. Low, West Lafayette, IN (US); Nimalka A. Bandara, Lafayette, IN (US)

(21) Appl. No.: 14/966,530

(22) Filed: Dec. 11, 2015

Related U.S. Application Data
(60) Provisional application No. 62/091,437, filed on Dec. 12, 2014.

(51) Int. Cl. A61K 39/385; A61K 31/403; A61K 31/4439; A61K 39/39; A61K 47/48

(52) U.S. Cl. CPC A61K 39/385; A61K 39/39; A61K 47/4833; A61K 47/48284; A61K 31/4439; A61K 31/403; A61K 2039/585

(57) ABSTRACT

The invention relates to a method of treating a cancer, the method comprising the steps of administering to a patient an immunogen conjugate, administering to the patient a folate conjugate comprising a folate linked to a hapten, and administering to the patient a tyrosine kinase inhibitor.

### CROSS-REFERENCE TO RELATED APPLICATIONS

This application claims priority under 35 U.S.C §119(e) to U.S. Provisional Application Ser. No. 62/091,437, filed on Dec. 12, 2014, the disclosure of which is herein incorporated by reference.

### FIELD OF THE DISCLOSURE

The invention relates to a method of treating a cancer, the method comprising the steps of administering to a patient an immunogen conjugate, administering to the patient a folate conjugate comprising a folate linked to a targeting hapten, and administering to the patient a tyrosine kinase inhibitor.

### BACKGROUND

The mammalian immune system provides a means for the recognition and elimination of cancer cells. While the immune system normally provides a strong line of defense, there are still many instances where cancer cells evade a host immune response and proliferate or persist with concomitant pathogenicity to the patient. Chemotherapeutic agents and radiation therapies have been developed to eliminate cancers. However, most, if not all, of the currently available chemotherapeutic agents and radiation therapy regimens have adverse side effects because they work not only to destroy cancer cells, but they also affect normal cells, such as cells of the hematopoietic system. The adverse side effects of the currently available anticancer drugs, highlight the need for the development of new therapies specific for cancer cells and with reduced toxicity to the patient.

Folate plays important roles in nucleotide biosynthesis and cell division, intracellular activities which occur in both malignant and certain normal cells. The folate receptor has a high affinity for folate, and folate, upon binding the folate receptor, impacts the cell cycle in dividing cells. As a result, folate receptors have been implicated in a variety of cancers (e.g., ovarian, endometrial, lung and breast) which have been shown to demonstrate high folate receptor expression. In contrast, folate receptor expression in normal tissues is limited (e.g., kidney, liver, intestines and placenta). This differential expression of the folate receptor in neoplastic and normal tissues makes the folate receptor a target for the development of anticancer therapies specific for cancer cells. There is a great need for methods to treat cancers using folate receptor-based targeting, along with methods to identify folate receptor positive cancers in patients.

### SUMMARY

In one embodiment, a method of treating a cancer in a patient in need of such a treatment is described. The method comprises the steps of administering to the patient a therapeutically effective amount of an immunogen conjugate, administering to the patient a therapeutically effective amount of a folate conjugate comprising a folate linked to a targeting hapten, and administering to the patient a therapeutically effective amount of a tyrosine kinase inhibitor.

Embodiments of the invention are further described by the following clauses:

1. A method of treating a cancer in a patient in need of such a treatment, the method comprising the steps of

administering to the patient a therapeutically effective amount of an immunogen conjugate,

administering to the patient a therapeutically effective amount of a folate conjugate comprising a folate linked to a targeting hapten, and

administering to the patient a therapeutically effective amount of a tyrosine kinase inhibitor.

2. The method of clause 1 wherein the cancer is a folate receptor expressing cancer.

3. The method of any one of clauses 1 to 2, wherein the cancer is selected from the group consisting of a carcinoma, a sarcoma, a lymphoma, a melanoma, a mesothelioma, a leukemia, an adenocarcinoma, and a myeloma.

4. The method of any one of clauses 1 to 3, wherein the cancer is selected from the group consisting of lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, oral cancer, laryngeal cancer, testicular cancer, liver cancer, non-small cell lung cancer, cancer of the adrenal gland, cancer of the urethra, prostate cancer, chronic leukemia, acute leukemia, lymphocytic lymphoma, pleural mesothelioma, nasopharyngeal carcinoma, cancer of the bladder, Burkitt's lymphoma, cancer of the ureter, kidney cancer, renal cell carcinoma, brain cancer, and pituitary adenoma.

5. The method of any one of clauses 1 to 4, wherein the cancer is non-small cell lung cancer.

6. The method of any one of clauses 1 to 4, wherein the cancer is ovarian cancer.

7. The method of any one of clauses 1 to 4, wherein the cancer is renal cell carcinoma.

8. The method of any one of clauses 1 to 7 wherein the immunogen conjugate comprises a carrier linked to an antigenic hapten.

9. The method of clause 8 wherein the immunogen conjugate has the formula

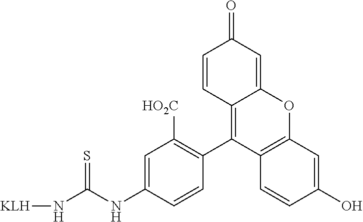

or a pharmaceutically acceptable salt thereof

10. The method of any one of clauses 1 to 9 wherein the folate has the formula

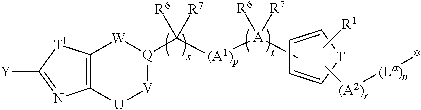

wherein X and Y are each-independently selected from the group consisting of halo, R², OR³, SR³, and NR⁴R⁵;

U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, NR^{8a}, and —(R⁸)C═C(R⁸)—; T¹ is —N═C(X)— or —NH—C(O)—;

A¹ and A² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;

R¹ is selected from the group consisting of hydrogen, halo, C₁-C₁₂ alkyl, and C₁-C₁₂ alkoxy; R², R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R³ is in each instance independently selected from the group consisting of hydrogen, C₁-C₁₂ alkyl, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁴, R^{4a}, R^{4b}, R⁵, and R^{5b} are each independently selected from the group consisting of hydrogen, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;

R^{8a} is hydrogen, C₁-C₁₂ alkyl, and C₁-C₁₂ alkoxy, or a bond to (A²)ᵣ-(L^{a})ₙ; R⁸ is independently selected in each instance from R¹ or a bond to (A²)ᵣ-(L^{a})ₙ;

L^{a} is a divalent linker as described herein;

n, p, r, s and t are each independently either 0 or 1; and

* denotes the attachment point to the remainder of the conjugate;

or a pharmaceutically acceptable salt thereof.

11. The method of any one of clauses 1 to 10 wherein the folate is folate.

12. The method of any one of clauses 1 to 11 wherein the antigenic hapten and the targeting hapten have the formula

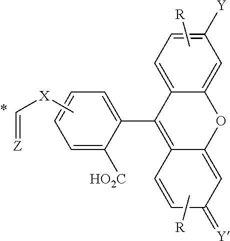

wherein X is O, NH, or S, and where * denotes the point of attachment of the hapten to the rest of the conjugate; Z is O or S; Y is OR^{a}, NR^{a}₂, or NR^{a}₃⁺; and Y′ is O, NR^{a}, or NR^{a}₂⁺, where R^{a} is hydrogen or C₁-C₆ alkyl; and each R independently represents from 0-2 substituents independently selected in each instance from the group consisting of halogen, hydroxyl, C₁-C₆ alkyl, C₁-C₆ alkyloxy, amino, C₁-C₆ alkylamino, (C₁-C₆ alkyl)₂amino, nitro, cyano, cyanate, thiocyanate, C₁-C₆ alkylcarbonyl, C₁-C₆ alkyloxycarbonyl, C₁-C₆ alkylaminocarbonyl, C₁-C₆ alkylcarbonylamino, C₁-C₆ alkylcarbonyloxy, C₁-C₆ alkylthio, SO₃H, and CO₂H; or a pharmaceutically acceptable salt thereof.

13. The method of any one of clauses 1 to 12 wherein the folate conjugate has the formula

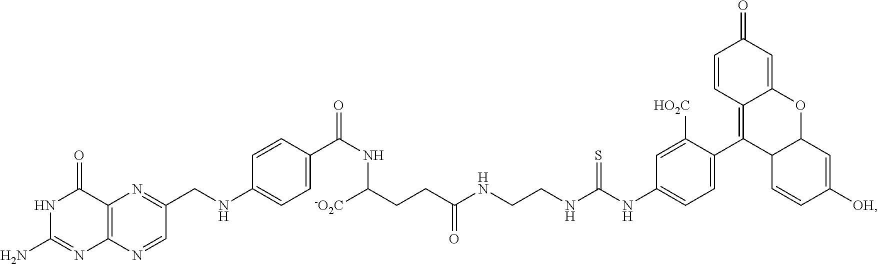

or a pharmaceutically acceptable salt thereof.

14. The method of any one of clauses 1 to 13 wherein the tyrosine kinase inhibitor has the formula

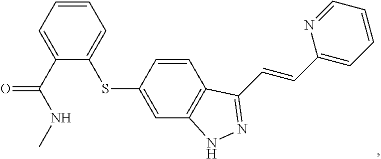 [SMILES: CNC(=O)c1ccccc1Sc1ccc2c(/C=C/c3ccccn3)n[nH]c2c1]

or a pharmaceutically acceptable salt thereof.

15. The method of any one of clauses 1 to 13 wherein the tyrosine kinase inhibitor has the formula

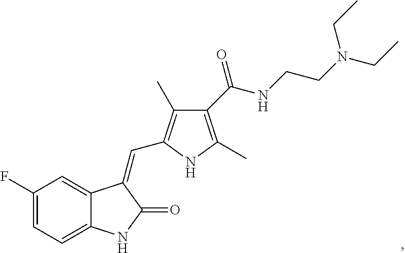 [SMILES: CCN(CC)CCNC(=O)c1c(C)[nH]c(/C=C2\C(=O)Nc3ccc(F)cc32)c1C]

or a pharmaceutically acceptable salt thereof.

16. The method of any one of clauses 1 to 15, wherein the immunogen conjugate, the folate conjugate, and/or the tyrosine kinase inhibitor are administered in a parenteral dosage form wherein the parenteral dosage form is selected from the group consisting of intradermal, subcutaneous, intramuscular, intraperitoneal, intravenous, and intrathecal.

17. The method of any one of clauses 1 to 16, wherein the therapeutically effective amount is from about 0.5 mg/m² to about 6.0 mg/m².

18. The method of any one of clauses 1 to 17, wherein the therapeutically effective amount is from about 0.5 mg/m² to about 5.0 mg/m².

19. The method of any one of clauses 1 to 18, wherein the therapeutically effective amount is from about 0.5 mg/m² to about 4.0 mg/m².

20. The method of any one of clauses 1 to 19, wherein the therapeutically effective amount is from about 0.5 mg/m² to about 3.5 mg/m².

21. The method of any one of clauses 1 to 20, wherein the therapeutically effective amount is from about 0.5 mg/m² to about 3.0 mg/m².

22. The method of any one of clauses 1 to 21, wherein the therapeutically effective amount is from about 0.5 mg/m² to about 2.5 mg/m².

23. The method of any one of clauses 1 to 22, wherein the therapeutically effective amount is from about 0.5 mg/m² to about 2.0 mg/m².

24. The method of any one of clauses 1 to 23, further comprising the step of detecting folate receptor expression by the cancer.

25. The method of clause 24, wherein the step of detecting occurs before any of the steps of administering.

26. The method of clause 24 or 25, wherein the detecting is performed by imaging and wherein the imaging is selected from the group consisting of SPECT imaging, PET imaging, IHC, and FISH.

27. The method of clause 26, wherein the detecting is performed by SPECT imaging.

28. The method of any one of clauses 26 to 27, wherein the step of detecting comprises administering to the patient an imaging conjugate of the formula

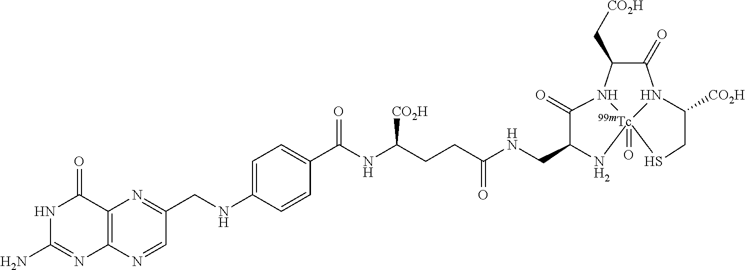

or a pharmaceutically acceptable salt thereof.

29. The method of any one of clauses 1 to 28 further comprising administering an adjuvant to the patient.

30. The method of clause 29 wherein the adjuvant is a saponin adjuvant.

31. The method of clause 30 wherein the adjuvant is a quillajasaponin adjuvant.

32. The method of clause 31 wherein quillajasaponin adjuvant is GPI-0100.

### BRIEF DESCRIPTION OF THE DRAWINGS

FIG. 1 is a plot showing the results of an endpoint Elisa used to determine levels of anti-FITC antibodies in immunized Balb/c mice. Serum collected from KLH-FITC immunized mice was assayed alongside serum from unimmunized mice.

FIG. 2 is a plot showing tumor volume over time in Renca tumor-bearing mice treated with PBS, folate-FITC alone, sunitinib alone, or a combination of folate-FITC and sunitinib. Folate-FITC immunotherapy with sunitinib performed better than either folate-FITC or sunitinib alone. In the figure, the line representing the results for the PBS control is the topmost line, the line representing the results for immunotherapy alone is the second topmost line, the line representing the results for the treatment with sunitinib alone is the second line from the bottom, and the line representing the results for the combination therapy is the bottom-most line, at the longest duration.

FIG. 3 is a plot showing a tritiated thymidine incorporation assay used to measure the IC50 of sunitinib on L1210A cells. Sunitinib only had weak cytotoxicity against these cells in vitro.

FIG. 4 is a plot showing tumor volume over time in L1210A tumor-bearing mice treated with PBS, folate-FITC alone, sunitinib alone, or a combination of folate-FITC and sunitinib. Treatment with the folate-FITC and sunitinib combination slows tumor growth and prolongs survival in DBA/2 mice implanted with L1210A tumors when compared to mice treated with either therapy alone. In the figure, the line representing the results for immunotherapy alone is the topmost line, the line representing the results for the treatment with sunitinib alone is the second topmost line, the line representing the results for the PBS control is the second line from the bottom, and the line representing the results for the combination therapy is the bottom-most line, at the longest duration.

FIG. 5 shows the percentage of CD4+ and CD8+ cytotoxic T cell in spleens of L1210A tumor-bearing mice treated with PBS, folate-FITC alone, sunitinib alone, or a combination of folate-FITC and sunitinib. The combination of folate-FITC with sunitinib increased both CD4+ and CD8+ cytotoxic T cell levels in treated L1210A tumor-bearing mice compared to mice treated with either therapy alone.

FIG. 6 shows the intra-tumoral vasculature content in L1210A tumor-bearing mice treated with PBS, folate-FITC alone, sunitinib alone, or a combination of folate-FITC and sunitinib. The folate-FITC with sunitinib combination therapy dramatically decreased intra-tumoral vasculature content compared to treatment with PBS or either treatment alone.

FIG. 7 shows images of excised tumors and spleens taken from L1210A tumor-bearing mice treated with PBS, folate-FITC alone, sunitinib alone, or a combination of folate-FITC and sunitinib.

FIG. 8 shows the average weights of tumors in L1210A tumor-bearing mice treated with PBS, folate-FITC alone, sunitinib alone, or a combination of folate-FITC and sunitinib, showing the comparative smallness of the tumors collected from the mice in the combination group.

FIG. 9 shows the average weights of spleens in L1210A tumor-bearing mice treated with PBS, folate-FITC alone, sunitinib alone, or a combination of folate-FITC and sunitinib, showing swelling of the spleens of tumor-bearing mice that were not treated with the combination therapy.

FIG. 10 shows images of excised tumors and spleens taken from M109 tumor-bearing mice treated with PBS, folate-FITC alone, sunitinib alone, or a combination of folate-FITC and sunitinib.

FIG. 11 is a plot showing tumor volume over time in M109 tumor-bearing mice treated with PBS, folate-FITC alone, sunitinib alone, or a combination of folate-FITC and sunitinib. Treatment with the folate-FITC and sunitinib combination synergized to slow tumor growth and prolongs survival in Balb/c mice implanted with M109 tumors when compared to mice treated with either therapy alone. In the figure, the line representing the results for the PBS control is the topmost line, the line representing the results for the treatment with sunitinib alone is the second topmost line, the line representing the results for immunotherapy alone is the second line from the bottom, and the line representing the results for the combination therapy is the bottom-most line, at the longest duration.

FIG. 12 shows the average weights of tumors in M109 tumor-bearing mice treated with PBS, folate-FITC alone, sunitinib alone, or a combination of folate-FITC and sunitinib, showing the comparative smallness of the tumors collected from the mice in the combination group.

FIG. 13 shows the percentage of F480+ macrophage cells in digested M109 tumors of M109 tumor-bearing mice treated with PBS, folate-FITC alone, sunitinib alone, or a combination of folate-FITC and sunitinib. Treatment with the folate-FITC and sunitinib combination increased the average F480+ macrophage population compared to mice treated with either therapy alone.

FIG. 14 shows the percentage of myeloid derived suppressor cells (MDSC) in the spleens of M109 tumor-bearing mice treated with PBS, folate-FITC alone, sunitinib alone, or a combination of folate-FITC and sunitinib. Treatment with the folate-FITC and sunitinib combination decreased the average MDSC population compared to mice treated with either therapy alone.

FIG. 15 shows the percentage of CD4+ and CD8+ cytotoxic T cell in spleens of M109 tumor-bearing mice treated with PBS, folate-FITC alone, sunitinib alone, or a combination of folate-FITC and sunitinib. The combination of folate-FITC with sunitinib increased both CD4+ and CD8+ cytotoxic T cell levels in treated M109 tumor-bearing mice compared to mice treated with either therapy alone.

FIG. 16 is a plot showing tumor volume over time in L1210A tumor-bearing mice treated with PBS, folate-FITC alone, axitinib alone, or a combination of folate-FITC and axitinib. Treatment with the folate-FITC and axitinib combination synergized to slow tumor growth and prolonged survival in DBA/2 mice implanted with L1210A tumors when compared to mice treated with either therapy alone. In the figure, the line representing the results for the PBS control is the topmost line, the line representing the results for immunotherapy alone is the second topmost line, the line representing the results for the treatment with axitinib alone is the second line from the bottom, and the line representing the results for the combination therapy is the bottom-most line, at the longest duration.

FIG. 17 shows the average weights of tumors in L1210A tumor-bearing mice treated with PBS, folate-FITC alone, axitinib alone, or a combination of folate-FITC and axitinib, showing the comparative smallness of the tumors collected from the mice in the combination group.

FIG. 18 shows the average weights of spleens in L1210A tumor-bearing mice treated with PBS, folate-FITC alone, axitinib alone, or a combination of folate-FITC and axitinib, showing swelling of the spleens of tumor-bearing mice that were not treated with the combination therapy.

### DETAILED DESCRIPTION OF THE ILLUSTRATIVE EMBODIMENTS

A method is provided of treating a patient with a cancer. In one embodiment, the method is based on increasing the immune system response to a cancer in a patient by increasing the antigenicity of the cancer to enhance an endogenous immune response-mediated reduction or elimination of the cancer cells. In one aspect, the patient is administered an immunogen conjugate, and then a folate conjugate comprising a folate linked to a targeting hapten is administered to the patient for binding to the cancer cells and the folate conjugates “label” the targeted cancer cells with the targeting hapten, thereby triggering an immune-mediated response directed at the cancer cells. In another aspect, antibodies existing or produced in the patient bind to the targeting hapten and trigger endogenous immune responses against the cancer. In another aspect, tyrosine kinase inhibitors act synergistically with the immunogen conjugates and/or the folate conjugates to enhance the immune response in the patient.

In accordance with the method described herein, the embodiments of the enumerated clauses provided in the Summary section above, or any combination thereof, are contemplated for combination with any of the embodiments described in the Detailed Description section of this patent application.

In one embodiment, a method of treating a cancer in a patient in need of such a treatment is described. The method comprises the steps of administering to the patient a therapeutically effective amount of an immunogen conjugate, administering to the patient a therapeutically effective amount of a folate conjugate comprising a folate linked to a targeting hapten, and administering to the patient a therapeutically effective amount of a tyrosine kinase inhibitor.

In one embodiment, the method is applicable to a folate receptor expressing cancer. In another embodiment, the cancer is selected from the group consisting of a carcinoma, a sarcoma, a lymphoma, a melanoma, a mesothelioma, a leukemia, an adenocarcinoma, and a myeloma. In still another embodiment, the cancer is selected from the group consisting of lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, cancer of the urethra, prostate cancer, chronic leukemia, acute leukemia, lymphocytic lymphoma, pleural mesothelioma, nasopharyngeal carcinoma, oral cancer, laryngeal cancer, testicular cancer, liver cancer, cancer of the bladder, Burkitt's lymphoma, cancer of the ureter, kidney cancer, renal cell carcinoma, brain cancer, and pituitary adenoma.

In other aspects, the cancer is non-small cell lung cancer, ovarian cancer, or renal cell carcinoma. In still other illustrative embodiments, the cancer can be tumorigenic, including benign tumors and malignant tumors, or it can be non-tumorigenic. In another illustrative aspect, the cancer can arise spontaneously or by such processes as mutations present in the germline of the patient or somatic mutations, or it may be chemically-, virally-, or radiation-induced.

In various embodiments, the method described herein can be used for both human clinical medicine and veterinary applications. Thus, the patient can be a human patient or, in the case of veterinary applications, may be a laboratory, agricultural, domestic, or wild animal. In other embodiments, the method is applicable to animals including, but not limited to, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

In one embodiment, an immunogen conjugate is administered to the patient. In one aspect, the immunogen conjugate can be a carrier (e.g., KLH or BSA) linked to an antigenic hapten. As used herein, “antigenic hapten” means a hapten that is antigenic and is therapeutically effective in the method described herein when attached to a carrier for administration to the patient according to the steps of the method described herein. In one aspect, the immunogen conjugate can be administered to the patient to elicit a preexisting immunity to the antigenic hapten and/or the targeting hapten. In another embodiment, an adjuvant is co-administered with the immunogen conjugate. In one aspect, the adjuvant enhances the immune response to the targeting hapten upon administration of the folate conjugate (i.e., a folate linked to a targeting hapten). As used herein, “targeting hapten” means the hapten linked to the folate wherein the immune response is directed to the targeting hapten associated with the cancer as a result of binding of the folate conjugate to the cancer cells.

For all of the embodiments described herein, “co-administration” is defined as administration at a time prior to, at the same time as, or at a time following administration of the immunogen conjugate or the folate conjugate. In one illustrative embodiment, “co-administration” can also mean administration in the same or different solutions.

In various embodiments, adjuvants suitable for use in accordance with the method described herein include, but are not limited to, saponin adjuvants (e.g., the quillajasaponins (e.g., GPI-0100)), CpG, 3-deacylated monophosphoryl lipid A (MPL), Bovine Calmette-Guerin (BCG), double stem-loop immunomodulating oligodeoxyribonucleotides (d-SLIM), heat-killed Brucella abortus (HKBA), heat-killed Mycobacterium vaccae (SRL172), inactivated vaccinia virus, cyclophosphamide, prolactin, thalidomide, actimid, revimid, and the like. Saponin adjuvants and methods of their preparation and use are described in detail in U.S. Pat. Nos. 5,057,540, 5,273,965, 5,443,829, 5,508,310, 5,583,112, 5,650,398, 5,977,081, 6,080,725, 6,231,859, and 6,262,029 incorporated herein by reference.

In one embodiment, administration of the immunogen conjugate can involve multiple injections of the immunogen conjugate. In one aspect, the adjuvant can be administered with the immunogen conjugate using any schedule, such as at a time prior to, at the same time as, or at a time following administration of an immunogen conjugate. In one illustrative embodiment, the adjuvant can be administered in the same solution or in a different solution than the immunogen conjugate.

In illustrative aspects, carriers that can be used in accordance with the method described herein include keyhole limpet hemocyanin (KLH), haliotis tuberculata hemocyanin (HtH), inactivated diptheria toxin, inactivated tetanus toxoid, purified protein derivative (PPD) of Mycobacterium tuberculosis, bovine serum albumin (BSA), ovalbumin (OVA), g-globulins, thyroglobulin, peptide antigens, and synthetic carriers, such as poly-L-lysine, dendrimer, liposomes, and other carriers known in the art.

In one embodiment, the carrier (e.g., KLH or BSA) can be linked to the antigenic hapten by using any art-recognized method of forming a conjugate. In various embodiments, this can include covalent, ionic, or hydrogen bonding of the carrier to the antigenic hapten, either directly or indirectly via a linking group such as a divalent linker. In one aspect, the immunogen conjugate is formed by covalent bonding through the formation of amide, ester or imino bonds between acid, aldehyde, hydroxy, amino, or hydrazo groups on the carrier and the antigenic hapten. In embodiments where a divalent linker is used, the divalent linker may comprise about 1 to about 30 carbon atoms, more typically about 2 to about 20 carbon atoms. Lower molecular weight divalent linkers (i.e., those having an approximate molecular weight of about 20 to about 500) may be employed. In another aspect, the divalent linker can comprise an indirect means for associating the carrier with the antigenic hapten, such as by connection through intermediary linkers, spacer arms, or bridging molecules.

In one illustrative aspect, the antigenic hapten can have the formula

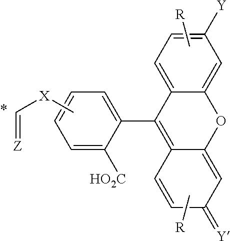

wherein X is O, NH, or S, and where * denotes the point of attachment of the hapten to the rest of the conjugate; Z is O or S; Y is OR^{a}, NR^{a}₂, or NR^{a}₃⁺; and Y′ is O, NR^{a}, or NR^{a}₂⁺, where R^{a} is hydrogen or C₁-C₆ alkyl; and each R independently represents from 0-2 substituents independently selected in each instance from the group consisting of halogen, hydroxyl, C₁-C₆ alkyl, C₁-C₆ alkyloxy, amino, C₁-C₆ alkylamino, (C₁-C₆ alkyl)₂amino, nitro, cyano, cyanate, thiocyanate, C₁-C₆ alkylcarbonyl, C₁-C₆ alkyloxycarbonyl, C₁-C₆ alkylaminocarbonyl, C₁-C₆ alkylcarbonylamino, C₁-C₆ alkylcarbonyloxy, C₁-C₆ alkylthio, SO₃H, and CO₂H; or a pharmaceutically acceptable salt thereof.

In another embodiment the antigenic hapten can have the formula

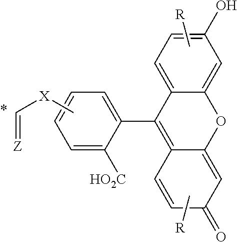

wherein X, Z, and R are as described herein.

In another embodiment the antigenic hapten can have the formula

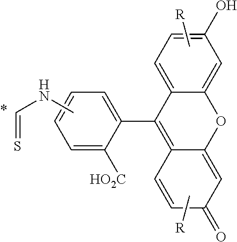

wherein R is as described herein.

In another embodiment, the immunogen conjugate can have the formula

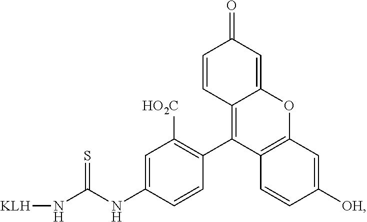

or a pharmaceutically acceptable salt thereof.

In one aspect, after a preexisting immunity is elicited, the folate conjugate (i.e., a folate linked to the targeting hapten) can be administered to the patient to “label” the cancer for an immune response against the cancer. In one aspect, the administration of the folate conjugate (i.e., a folate linked to the targeting hapten) can result in an humoral or cell-mediated immune response, or both, directed against the targeting hapten associated with the cancer.

In one illustrative aspect, the preexisting immunity can be an innate immunity against the targeting hapten and administration of the immunogen conjugate is optional, but may enhance the immune response against the cancer. In another embodiment, there may be no preexisting immunity, and the folate conjugate and passively administered antibodies can be co-administered, optionally with an adjuvant. In this embodiment, the passively administered antibodies help to augment the immune response to the cancer.

Illustrative embodiments of “a folate” as described herein for use in the folate conjugates (i.e., a folate linked to a hapten) include folate and analogs of folate such as folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refer to the art-recognized analogs having a carbon atom substituted for one or two nitrogen atoms in the naturally occurring folic acid structure, or analog or derivative thereof. For example, the deaza analogs include the 1-deaza, 3-deaza, 5-deaza, 8-deaza, and 10-deaza analogs of folate, folinic acid, pteropolyglutamic acid, and folate receptor-binding pteridines such as tetrahydropterins, dihydrofolates, and tetrahydrofolates. The dideaza analogs include, for example, 1,5-dideaza, 5,10-dideaza, 8,10-dideaza, and 5,8-dideaza analogs of folate, folinic acid, pteropolyglutamic acid, and folate receptor-binding pteridines such as tetrahydropterins, dihydrofolates, and tetrahydrofolates. In other embodiments, a folate can include the folate receptor-binding analogs aminopterin, amethopterin (also known as methotrexate), N¹⁰-methylfolate, 2-deamino-hydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate). In one aspect, the foregoing can bind with folate-receptors, and when linked to exogenous molecules are effective to enhance transmembrane transport, such as via folate-mediated endocytosis.

Other embodiments of “a folate” include folate and additional analogs of folic acid that bind to folic acid receptors and are described in U.S. Patent Application Publication Nos. 2005/0227985 and 2004/0242582, the disclosures of which are incorporated herein by reference. Illustratively, a folate can have the general formula:

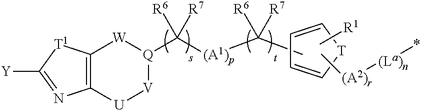

wherein X and Y are each-independently selected from the group consisting of halo, R², OR³, SR³, and NR⁴R⁵;

U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, NR^{8a}, and —(R⁸)C═C(R⁸)—; T′ is —N═C(X)— or —NH—C(O)—;

A¹ and A² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R⁶⁶)(R^{7b})—, —N(C≡CH)—, —N(CH₂C≡CH)—, C₁-C₁₂ alkylene, and C₁-C₁₂ alkyeneoxy, where Z is oxygen or sulfur;

R¹ is selected from the group consisting of hydrogen, halo, C₁-C₁₂, alkyl, and C₁-C₁₂ alkoxy; R², R^{6b}, and R^{7b} are each independently selected from the group consisting of hydrogen, halo, C₁-C₁₂ alkyl, C₁-C₁₂, alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R³ is in each instance independently selected from the group consisting of hydrogen, C₁-C₁₂ alkyl, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁴, R^{4a}, R^{4b}, R⁵, and R^{5b} are each independently selected from the group consisting of hydrogen, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂, alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;

R^{8a} is hydrogen, C₁-C₁₂ alkyl, and C₁-C₁₂ alkoxy, or a bond to (A²)ᵣ-(L^{a})ₙ; R⁸ is independently selected in each instance from R¹ or a bond to (A²)ᵣ-(L^{a})ₙ;

L^{a} is a divalent linker as described herein;

n, p, r, s and t are each independently either 0 or 1; and

* denotes the attachment point to the remainder of the conjugate.

In one aspect of such a folate, when s is 1, t is 0, and when s is 0, t is 1. In another aspect of such folate and folate analogs, both n and r are 1, and linker L^{a} includes a naturally occurring amino acid covalently linked to A² at its alpha-amino group through an amide bond. Illustrative amino acids include aspartic acid, glutamic acid, lysine, cysteine, and the like. In another aspect of such a folate s is 1, t is 0, T¹ is —NH—C(O)—; T is —HC═C(A²)-; A¹ is NH; and Y is NH₂.

As used herein, it is to be understood that the term “a folate” refers both individually to folate that forms a folate conjugate, or alternatively to a folate analog, that is capable of binding to folate receptors or folic acid receptors.

Illustrative targeting haptens that can be part of the folate conjugate have the formula:

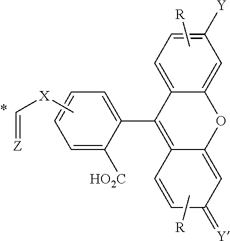

wherein X is O, NH, or S, and where * shows the point of attachment of the hapten to the rest of the compound; Z is O or S; Y is OR^{a}, NR^{a}₂, or NR^{a}₃⁺; and Y′ is O, NR^{a}, or NR^{a}₂⁺, where R^{a} is hydrogen or C₁-C₆ alkyl; and each R independently represents from 0-2 substituents independently selected in each instance from the group consisting of halogen, hydroxyl, C₁-C₆ alkyl, C₁-C₆ alkyloxy, amino, C₁-C₆ alkylamino, (C₁-C₆ alkyl)₂amino, nitro, cyano, C₁-C₆ alkylcarbonyl, C₁-C₆ alkyloxycarbonyl, C₁-C₆ alkylaminocarbonyl, C₁-C₆alkylcarbonylamino, C₁-C₆ alkylcarbonyloxy, C₁-C₆ alkylthio, SO₃H, or a salt thereof, CO₂H, or a salt thereof, and the like.

In another embodiment the targeting hapten can have the formula

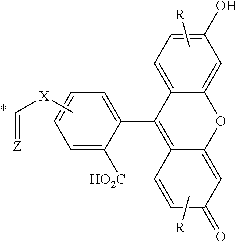

wherein X, Z, and R are as described herein.

In another embodiment the targeting hapten can have the formula

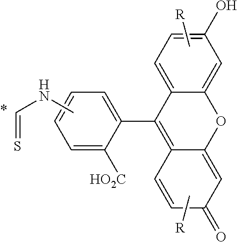

wherein R is as described herein.

In one embodiment, a folate in the folate conjugate can be conjugated to the targeting hapten by a procedure that utilizes trifluoroacetic anhydride to prepare γ-esters of folic acid via a pteroyl azide intermediate. This procedure results in the synthesis of a folate, conjugated to the targeting hapten only through the γ-carboxy group of the glutamic acid groups of folate wherein the γ-conjugate binds to the folate receptor with high affinity, avoiding the formation of mixtures of an α-conjugate and the γ-conjugate. Alternatively, pure α-conjugates can be prepared from intermediates wherein the γ-carboxy group is selectively blocked, the α-conjugate is formed and the γ-carboxy group is subsequently deblocked using art-recognized organic synthesis protocols and procedures.

In another embodiment, a folate can be linked to the targeting hapten by using any art-recognized method of forming a conjugate. In various embodiments, this can include covalent, ionic, or hydrogen bonding of a folate to the targeting hapten, either directly or indirectly via a linking group such as a divalent linker. In one aspect, the folate conjugate is formed by covalent bonding through the formation of amide, ester or imino bonds between acid, aldehyde, hydroxy, amino, or hydrazo groups on a folate and the targeting hapten. In embodiments where a divalent linker is used, the divalent linker may comprise about 1 to about 30 carbon atoms, more typically about 2 to about 20 carbon atoms. Lower molecular weight divalent linkers (i.e., those having an approximate molecular weight of about 20 to about 500) may be employed. In another aspect, the divalent linker can comprise an indirect means for associating a folate with the targeting hapten, such as by connection through intermediary linkers, spacer arms, or bridging molecules. In some embodiments, the divalent linker can be a diaminoalkyl group, such as ethylenediamine, propylenediamine, butylenediamine, and the like.

In some embodiments, the divalent linker comprises the formula

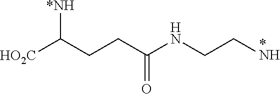

wherein each * represents a covalent bond to either folate or FITC.

In some embodiments, the divalent linker comprises the formula

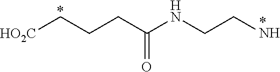

wherein each * represents a covalent bond to either folate or FITC.

In one illustrative aspect, the folate conjugate is folate-FITC (also referred to herein and in the art as EC17) having the formula

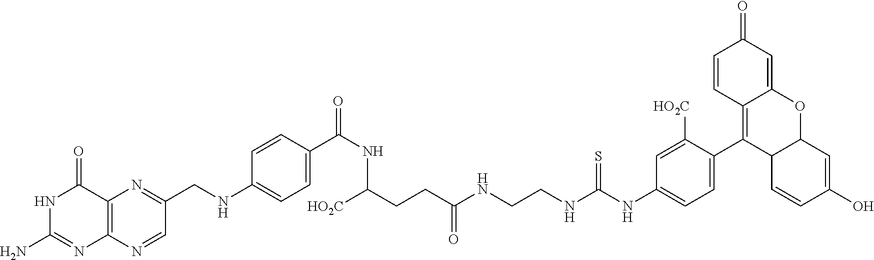 [SMILES: Nc1nc2ncc(CNc3ccc(C(=O)NC(CCC(=O)NCCNC(=S)Nc4ccc(C5=C6C=CC(=O)C=C6OC6C=C(O)C=CC56)c(C(=O)O)c4)C(=O)O)cc3)nc2c(=O)[nH]1]

or a pharmaceutically acceptable salt thereof.

In some embodiments, the conjugate has the formula

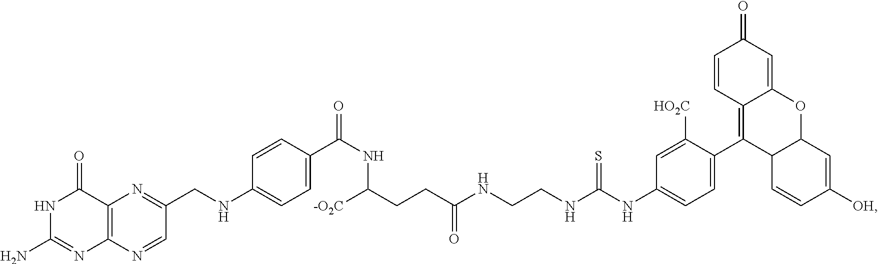

or a pharmaceutically acceptable salt thereof.

In another illustrative aspect, combinations of immunogen conjugates and folate conjugates (i.e., a folate linked to a hapten) can be used in the method described herein. In another method embodiment, a tyrosine kinase inhibitor is co-administered with the folate conjugate (i.e., a folate linked to a hapten) and/or the immunogen conjugate and can act synergistically with the folate conjugate and/or the immunogen conjugate to decrease tumor (i.e., the cancer) volume, slow tumor growth, prolong survival of the patient, decrease MDSC cells, inhibit tumor vasculature, and/or increase the number of circulating immune cells (e.g., CD4+ or CD8+ cells or F4/80+), for example.

In one embodiment, the tyrosine kinase inhibitor has the formula

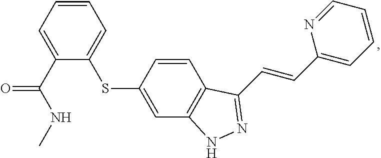 [SMILES: CNC(=O)c1ccccc1Sc1ccc2c(/C=C/c3ccccn3)n[nH]c2c1]

or a pharmaceutically acceptable salt thereof.

In another embodiment, the tyrosine kinase inhibitor has the formula

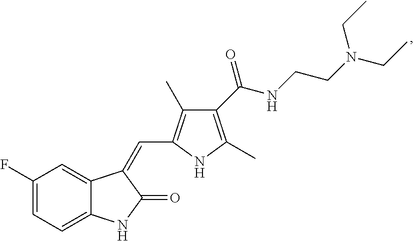 [SMILES: CCN(CC)CCNC(=O)c1c(C)[nH]c(/C=C2\C(=O)Nc3ccc(F)cc32)c1C]

or a pharmaceutically acceptable salt thereof.

In one illustrative aspect, the tyrosine kinase inhibitor can be co-administered with the folate conjugate. In one embodiment, the tyrosine kinase inhibitor can be administered to the patient prior to, after, or at the same time as the folate conjugate and the tyrosine kinase inhibitor can be administered as part of the same composition containing the folate conjugate or as part of a different composition than the folate conjugate.

It is to be understood that the compounds described herein may exist in one or more tautomeric forms. It is to be further understood that all tautomeric forms of the compounds described herein are contemplated.

In various illustrative aspects, the endogenous immune response that acts against the cancer can include an humoral response, a cell-mediated immune response, and any other immune response endogenous to the patient, including complement-mediated cell lysis, antibody-dependent cell-mediated cytoxicity (ADCC), antibody opsonization leading to phagocytosis, clustering of receptors upon antibody binding resulting in signaling of apoptosis, antiproliferation, or differentiation, and direct immune cell recognition of the delivered targeting hapten. In another aspect, the endogenous immune response may employ the secretion of cytokines that regulate such processes as the multiplication and migration of immune cells. In one illustrative aspect, the endogenous immune response can include the participation of such immune cell types as B cells, T cells, including helper and cytotoxic T cells, macrophages, natural killer cells, neutrophils, LAK cells, and the like.

In one aspect, it is contemplated that the preexisting antibodies (i.e., an innate immunity), induced antibodies, or passively administered antibodies will be redirected to the cancer by preferential binding of the folate conjugates to the cancer cells. In one illustrative embodiment, the method can also involve secondary responses that arise when the attracted antigen-presenting cells phagocytose or attack the cancer cells and present natural tumor antigens to the cellular arm of the immune system for elimination or reduction of the cancer cells bearing the antigens.

In one embodiment, a method as described in clause 1 above is described further comprising the step of detecting folate receptor expression by the cancer. In this embodiment, the step of detecting can occur before any of the steps of administering. In one aspect of the method wherein the detecting step is performed, the detecting can be by imaging wherein the imaging is selected from the group consisting of SPECT imaging, PET imaging, IHC, and FISH. In one illustrative aspect, the detecting is performed by SPECT imaging. In another embodiment, the step of detecting comprises administering to the patient an imaging conjugate of the formula

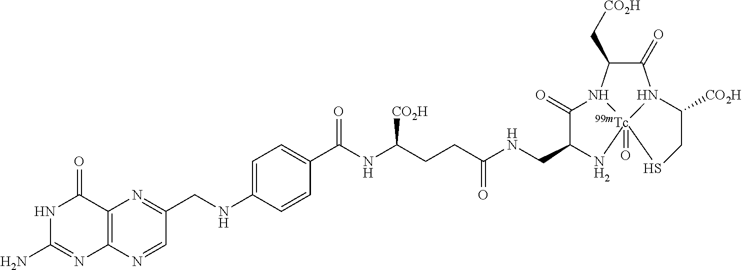

or a pharmaceutically acceptable salt thereof. If the patient is folate receptor positive as determined by a variety of methods, including those known in the art, the method described herein may be indicated for the patient. In other embodiments, any of a variety of folate-imaging agent conjugates detectable by PET imaging, SPECT imaging, and the like can be used. The exact manner of imaging is not limited to the imaging agents described herein.

In this method embodiment, for detecting by imaging, unlabeled folic acid, or a pharmaceutically acceptable salt thereof, can be co-administered to the patient with the folate-imaging agent conjugate, or a pharmaceutically acceptable salt thereof.

In various embodiments, therapeutically effective amounts of the adjuvant, immunogen conjugates, folate conjugates, and tyrosine kinase inhibitors, described herein include amounts ranging from about 0.1 MIU/m²/dose/day to about 60 MIU/m²/dose/day and about 0.1 MIU/m²/dose/day to about 10 MIU/m²/dose/day (MIU=million international units; m²=approximate body surface area of an average human).

In other embodiments, therapeutically effective amounts of the adjuvant, immunogen conjugates, folate conjugates, and tyrosine kinase inhibitors, can vary depending on the host condition, the cancer being treated, the molecular weight of the adjuvant, immunogen conjugates, folate conjugates, and tyrosine kinase inhibitors being used, the route of administration and tissue distribution, and the possibility of co-usage of other therapeutic treatments such as radiation therapy. In one aspect, the therapeutically effective amounts to be administered to a patient are based on body surface area, patient weight, and physician assessment of patient condition. In various illustrative aspects, therapeutically effective amounts can range from about 1 ng/kg to about 1 mg/kg, about 1 ng/kg to about 10 mg/kg, about 1 ng/kg to about 100 mg/kg, or from about 1 μg/kg to about 50 mg/kg, or from about 1 μg/kg to about 30 μg/kg, or from about 1 μg/kg to about 20 μg/kg, wherein “kg” is kilograms of patient body weight. The administration can occur, for example, in one or multiple daily doses or in a staggered dosage regimen.

In other embodiments, a therapeutically effective amount of the immunogen conjugates, folate conjugates, tyrosine kinase inhibitors or the adjuvants described herein can range, for example, from about 0.5 mg/m² to about 10.0 mg/m². The therapeutically effective amounts described herein also include ranges of about 0.5 m g/m² to about 9.5 mg/m², about 0.5 mg/m² to about 9.0 mg/m², about 0.5 mg/m² to about 8.5 mg/m², about 0.5 mg/m² to about 8.0 mg/m², about 0.5 mg/m² to about 7.5 mg/m², about 0.5 mg/m² to about 7.0 mg/m², about 0.5 mg/m² to about 6.5 mg/m², about 0.5 mg/m² to about 6.0 mg/m², about 0.5 mg/m² to about 5.5 mg/m², about 0.5 mg/m² to about 5.0 mg/m², about 0.5 mg/m² to about 4.5 mg/m², about 0.5 mg/m² to about 4.0 mg/m², about 0.5 mg/m² to about 3.5 mg/m², about 0.5 mg/m² to about 3.0 mg/m², about 0.5 mg/m² to about 2.5 mg/m², about 0.5 mg/m² to about 2.0 mg/m², about 0.5 mg/m² to about 1.5 mg/m², about 1.0 mg/m² to about 9.5 mg/m², about 1.0 mg/m² to about 9.0 mg/m², about 1.0 mg/m² to about 8.5 mg/m², about 1.0 mg/m² to about 8.0 mg/m², about 1.0 mg/m² to about 7.5 mg/m², about 1.0 mg/m² to about 7.0 mg/m², about 1.0 mg/m² to about 6.5 mg/m², about 1.0 mg/m² to about 6.0 mg/m², about 1.0 mg/m² to about 5.5 mg/m², about 1.0 mg/m² to about 5.0 mg/m², about 1.0 mg/m² to about 4.5 mg/m², about 1.0 mg/m² to about 4.0 mg/m², about 1.0 mg/m² to about 3.5 mg/m², about 1.0 mg/m² to about 3.0 mg/m², about 1.0 mg/m² to about 2.5 mg/m², about 1.0 mg/m² to about 2.0 mg/m², and about 1.0 mg/m² to about 1.5 mg/m². In one aspect, the therapeutically effective amount for any particular patient or group of patients may be any number value between about 0.5 mg/m² and about 10.0 mg/m², including but not limited to 1.0 mg/m², 1.5, mg/m², 2.0 mg/m², 2.5 mg/m², 3.0 mg/m², 3.5 mg/m², 4.0 mg/m², 4.5 mg/m², 5.0 mg/m², 5.5 mg/m², 6.0 mg/m², 6.5 mg/m², 7.0 mg/m², 7.5 mg/m², 8.0 mg/m², 8.5 mg/m², 9.0 mg/m², 9.5 mg/m² and 10.0 mg/m². In another aspect, the total dose may be administered in single or divided doses and may, at the physician's discretion, fall outside of the ranges given herein. In these embodiments, “m” means patient body mass. In another embodiment, the imaging conjugate can be administered in any of the amounts described herein.

In accordance with the method described herein “elimination” of cancer cells, tumor (i.e. cancer) cells, or tumor mass means a partial or complete elimination or lack of growth of the tumor. In one embodiment, the elimination can be an elimination of cells of the primary tumor or of cells that have metastasized or are in the process of dissociating from the primary tumor. In one aspect, a prophylactic treatment to prevent return of a cancer after its removal by any therapeutic approach including surgical removal of the cancer, radiation therapy, chemotherapy, or biological therapy is also contemplated.

In various embodiments, any effective regimen for administering the adjuvant, immunogen conjugate, folate conjugate, imaging conjugate, and/or tyrosine kinase inhibitor can be used. For example, the adjuvant, immunogen conjugate, folate conjugate, imaging conjugate, and/or tyrosine kinase inhibitor can be administered as single doses, or they can be divided and administered as a multiple-dose daily regimen. In other embodiments, a staggered regimen, for example, one to three days per week can be used as an alternative to daily treatment. In one embodiment, the patient is treated with multiple injections of the folate conjugate and the tyrosine kinase inhibitor, after three initial doses of the adjuvant and immunogen conjugate, to eliminate the cancer. In another embodiment, the patient is injected multiple times (e.g., about 2 up to about 50 times) with the folate conjugate, for example, at 12-72 hour intervals or at 48-72 hour intervals. In another aspect, additional injections of the folate conjugate can be administered to the patient at an interval of days or months after the initial injections(s) and the additional injections can reduce recurrence of disease. Alternatively, the initial injection(s) may prevent recurrence of the cancer. In another embodiment, the adjuvant, the immunogen conjugate, the folate conjugate, and/or the tyrosine kinase inhibitor can be administered daily. In yet another embodiment, the adjuvant, the immunogen conjugate, the folate conjugate, and/or the tyrosine kinase inhibitor can be administered in a single daily dose administered five days a week, in weeks 1, 2, 3, and 4 of each 4 week cycle. In an alternative example, the adjuvant, the immunogen conjugate, the folate conjugate, and/or the tyrosine kinase inhibitor can be administered in a single daily dose administered three days a week, of weeks 1, and 3 of each 4 week cycle, with no dose administered in weeks 2 and 4. In an alternative example, the adjuvant, the immunogen conjugate, the folate conjugate, and/or the tyrosine kinase inhibitor can be administered biweekly on weeks 1 and 2, i.e. on days 1, 4, 8, 11, of a 3-week cycle. In an alternative example, the adjuvant, the immunogen conjugate, the folate conjugate, and/or the tyrosine kinase inhibitor can be administered once weekly on weeks 1 and 2, i.e. days 1 and 8 of a 3-week cycle.

In one embodiment, a folate conjugate and/or immunogen conjugate that have a different targeting hapten and antigenic hapten, respectively, can be used wherein the targeting hapten and the antigenic hapten may be different, but both have the formula:

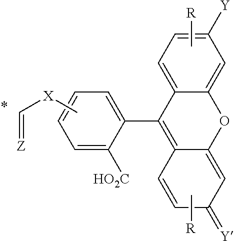

wherein X is O, NH, or S, and where * shows the point of attachment of the hapten to the rest of the compound; Z is O or S; Y is OR^{a}, NR^{a}₂, or NR^{a}₃⁺; and Y′ is O, NR^{a}, or NR^{a}₂⁺, where R^{a} is hydrogen or C₁-C₆ alkyl; and each R independently represents from 0-2 substituents independently selected in each instance from the group consisting of halogen, hydroxyl, C₁-C₆ alkyl, C₁-C₆ alkyloxy, amino, C₁-C₆ alkylamino, (C₁-C₆ alkyl)₂amino, nitro, cyano, C₁-C₆ alkylcarbonyl, C₁-C₆ alkyloxycarbonyl, C₁-C₆alkylaminocarbonyl, C₁-C₆alkylcarbonylamino, C₁-C₆ alkylcarbonyloxy, C₁-C₆alkylthio, SO₃H, or a salt thereof, CO₂H, or a salt thereof, and the like. In another embodiment, the folate conjugate and the immunogen conjugate may have a targeting hapten and an antigenic hapten, respectively, that have the same structure within the formula described in this paragraph. In another embodiment, the patient may be treated with multiple immunogen conjugates and folate conjugates in a co-dosing protocol.

In another embodiment the targeting hapten and/or the antigenic hapten can have the formula

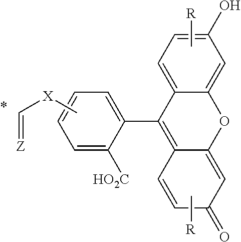

wherein X, Z, and R are as described herein.

In another embodiment the targeting hapten and/or the antigenic hapten can have the formula

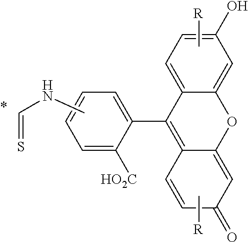

wherein R is as described herein.

In one aspect, the method described herein can be used in combination with additional therapies such as surgical removal of a tumor, radiation therapy, chemotherapy, or biological therapies such as other immunotherapies including, but not limited to, monoclonal antibody therapy, treatment with immunomodulatory agents, adoptive transfer of immune effector cells, treatment with hematopoietic growth factors, cytokines and vaccination.

In one embodiment, the adjuvant, the immunogen conjugate, the folate conjugate, the imaging conjugate, and/or the tyrosine kinase inhibitor are injected parenterally and such injections can be intradermal injections, intraperitoneal injections, subcutaneous injections, intramuscular injections, intravenous injections, or intrathecal injections, for example. In another embodiment, the adjuvant, the immunogen conjugate, the folate conjugate, the imaging conjugate, and/or the tyrosine kinase inhibitor are administered orally.

Examples of parenteral dosage forms include aqueous solutions of the active agent, in an isotonic saline, glucose or other well-known pharmaceutically acceptable liquid carriers such as liquid alcohols, glycols, esters, and amides. In one embodiment, the dosage form can be in the form of a reconstitutable or a reconstituted lyophilizate. In one embodiment, any of a number of prolonged release dosage forms known in the art can be administered such as, for example, the biodegradable carbohydrate matrices described in U.S. Pat. Nos. 4,713,249; 5,266,333; and 5,417,982, the disclosures of which are incorporated herein by reference. In another embodiment a slow pump can be used.

In other embodiments of the methods described herein, pharmaceutically acceptable salts of the adjuvant, immunogen conjugate, folate conjugate, the imaging conjugate, and/or the tyrosine kinase inhibitor can be used. Pharmaceutically acceptable salts may include acid addition and base salts.

In one aspect, suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

In another aspect, suitable base salts of the adjuvant, immunogen conjugate, folate conjugate, imaging conjugate, and/or the tyrosine kinase inhibitor described herein are formed from bases which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts.

In one embodiment, the adjuvant, immunogen conjugate, folate conjugate, imaging conjugate, and/or the tyrosine kinase inhibitor described herein may be administered as a formulation in association with one or more pharmaceutically acceptable carriers. The carriers can be excipients. The choice of carrier will to a large extent depend on factors such as the particular mode of administration, the effect of the carrier on solubility and stability, and the nature of the dosage form. Pharmaceutical compositions suitable for the delivery of the adjuvant, immunogen conjugate, folate conjugate, imaging conjugate, and/or the tyrosine kinase inhibitor described herein and methods for their preparation will be readily apparent to those skilled in the art. Such compositions and methods for their preparation may be found, for example, in Remington: The Science & Practice of Pharmacy, 21th Edition (Lippincott Williams & Wilkins, 2005), incorporated herein by reference.

In one illustrative aspect, a pharmaceutically acceptable carrier includes any and all solvents, dispersion media, coatings, antibacterial and antifungal agents, isotonic and absorption delaying agents, and the like, and combinations thereof, that are physiologically compatible. In some embodiments, the carrier is suitable for parenteral administration or oral administration. Pharmaceutically acceptable carriers include sterile aqueous solutions or dispersions and sterile powders for the extemporaneous preparation of sterile injectable or orally administrated solutions or dispersions.

In one embodiment, an aqueous suspension may contain the active materials in admixture with appropriate excipients. Such excipients can be suspending agents, for example, sodium carboxymethylcellulose, methylcellulose, hydroxypropylmethylcellulose, sodium alginate, polyvinylpyrrolidone, gum tragacanth and gum acacia; dispersing or wetting agents which may be a naturally-occurring phosphatide, for example, lecithin; a condensation product of an alkylene oxide with a fatty acid, for example, polyoxyethylene stearate; a condensation product of ethylene oxide with a long chain aliphatic alcohol, for example, heptadecaethyleneoxycetanol; a condensation product of ethylene oxide with a partial ester derived from fatty acids and a hexitol such as polyoxyethylene sorbitol monooleate; or a condensation product of ethylene oxide with a partial ester derived from fatty acids and hexitol anhydrides, for example, polyoxyethylene sorbitan monooleate. The aqueous suspensions may also contain one or more preservatives, for example, ascorbic acid, ethyl, n-propyl, or p-hydroxybenzoate; or one or more coloring agents.

Illustrative formats for oral administration include tablets, capsules, elixirs, syrups, and the like. It is appreciated that the adjuvant, immunogen conjugate, folate conjugate, imaging conjugate, and/or the tyrosine kinase inhibitor described herein may exist in unsolvated forms as well as solvated forms, including hydrated forms. In general, the solvated forms are equivalent to unsolvated forms and are encompassed within the scope of the present invention.

In another embodiment, compositions and/or dosage forms for administration of the adjuvant, immunogen conjugate, folate conjugate, imaging conjugate, and/or the tyrosine kinase inhibitor are prepared with a purity of at least about 90%, or about 95%, or about 96%, or about 97%, or about 98%, or about 99%, or about 99.5%. In another embodiment, compositions and or dosage forms have a purity of at least 90%, or at least 95%, or at least 96%, or at least 97%, or at least 98%, or at least 99%, or at least 99.5%.

As used herein, purity determinations may be based on weight percentage, mole percentage, and the like. In addition, purity determinations may be based on the absence or substantial absence of certain predetermined components. It is also to be understood that purity determinations are applicable to solutions of the adjuvant, immunogen conjugate, folate conjugate, imaging conjugate, and/or the tyrosine kinase inhibitor described herein. In those instances, purity measurements, including weight percentage and mole percentage measurements, are related to the components of the solution exclusive of the solvent. The purity may be measured using any conventional technique, including various chromatography or spectroscopic techniques, such as high pressure or high performance liquid chromatography (HPLC), nuclear magnetic resonance spectroscopy, TLC, UV absorbance spectroscopy, fluorescence spectroscopy, and the like.

In another embodiment, the method described herein may include the following examples. The examples further illustrate additional features of the various embodiments described herein. However, it is to be understood that the examples are illustrative and are not to be construed as limiting other embodiments described herein. In addition, it is appreciated that other variations of the examples are included in the various embodiments described herein.

### Materials for Immunization, Tumor Implantation, and Treatment:

Keyhole limpet hemocyanin (KLH)-FITC (KLH from Sigma Aldrich, St. Louis, Mo.), GPI-0100 (Endocyte, Inc., West Lafayette, Ind.), Saline (Sigma Aldrich, St. Louis, Mo.), Sterile PBS (Sigma Aldrich, St. Louis, Mo.), Folate-FITC (EC17, Endocyte, Inc., West Lafayette, Ind.), Sunitinib malate (LC Laboratories, Woburn, Mass.), Axitinib, free base (LC Laboratories, Woburn, Mass.),

### Treatment Schedule:

| Treatment | Dose (range tested) | Route | Frequency |
|---|---|---|---|
| PBS (for control) | 100 μL | S.C. | Daily |
| EC17 | 500 nmols/Kg | S.C. | Daily |
| | (500-1500 nmols/Kg) | | |
| Sunitinib malate | 20 mg/Kg | P.O. (Oral) | Daily |
| | (5-20 mg/Kg) | | |
| Axitinib, | 15 mg/Kg | P.O. | Daily |
| free base | (2-20 mg/Kg) | | |
| KLH-FITC | 35 mg KLH-FITC in | S.C. | Every 14 days for |
| | 50 mg GPI-0100 | | a total of 3 |
| | | | immunizations |

### Drug Formulations:

KLH-FITC is synthesized according to a previously published protocol (Y. Lu, 2000).

EC17 for treatment is prepared by diluting the stock solution obtained from Endocyte, Inc. in sterile PBS

Sunitinib for oral dosing is prepared by dissolving the drug in a carboxymethylcellulose solution according to protocols described in the literature (carboxymethylcellulose 0.5%, NaCl 1.8%, Tween 80 0.4%, and benzyl alcohol 0.9% in distilled water with pH adjusted to 6.0)

Axitinib for oral dosing is prepared by dissolving the drug in a carboxymethylcellulose solution according to protocols described in the literature (carboxymethylcellulose 0.5% in acidified (pH 2-3) distilled water)

Solid L1210A and Renca tumors are implanted by injecting 1×106 cells in serum free medium or sterile PBS subcutaneously on the left shoulder Solid M109 tumors are implanted by injecting 1×106 cells (at passage 0 or 1) suspended in sterile PBS containing 1% female Balb/c serum subcutaneously on the left shoulder

### Example 1

### Sunitinib/Folate-FITC Combination Therapy in Metastatic Renal Cell Carcinoma

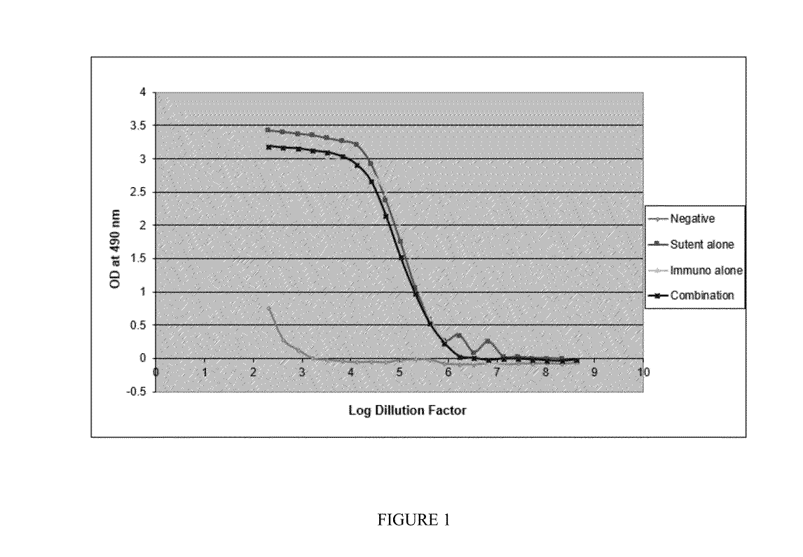

In order to test the effect of combining Sunitinib with folate-targeted immunotherapy on tumor regression in a clinically representative model, the combination therapy was evaluated in Renca tumors, a mouse syngeneic kidney cancer cell line. Renca cells were maintained in a folate deficient cell culture medium and were selected for high folate receptor (FR) expression via multiple steps of cell sorting before being used in therapeutic studies. Keyhole limpet hemocyanin (KLH) proteins conjugated to fluorescein isothiocyanate (FITC) were used as a vaccine agent to elicit an immune response leading to the production of autologous antibodies in study animals. In turn, folate-FITC was used as a folate-hapten conjugate in the therapeutic studies. Female Balb/c mice aged 5-6 weeks were purchased from Harlan Laboratories in Indianapolis, Ind. After allowing one week for the mice to acclimate to their environment, they were vaccinated with 35 ug KLH-FITC in 50 ug GPI-0100 as adjuvant. Mice were immunized every two weeks using subcutaneous injections at the base of tail or base of neck for a total of three vaccinations. Antibody titers were evaluated by enzyme-linked immunosorbent assay (ELISA) one week following the second and last immunizations to ensure that high levels of anti-FITC antibodies were being produced. A representative graph of the antibody titers produced in immunized Balb/c mice is shown in FIG. 1.

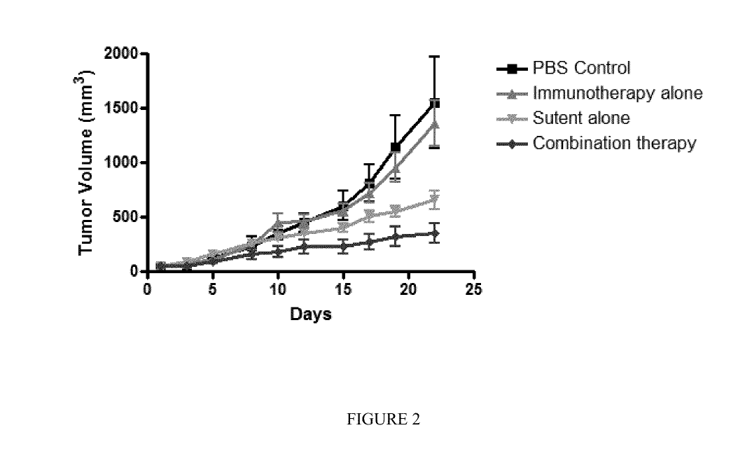

Several days before the third immunization, each mouse was injected subcutaneously on the left flank with 1×10⁶ Renca cells in serum free medium. When tumors were palpable, the mice were randomized into 4 groups: control, immunotherapy alone, sunitinib alone and sunitinib+immunotherapy (combination). Treatment was initiated when tumors measured 50 mm³. The control mice were treated with 100 uL of saline by intra peritoneal injection on Monday, Wednesday, and Friday. The immunotherapy mice were treated with 1500 nmols/Kg of folate-FITC in phosphate buffered saline (PBS) by intraperitoneal injection on Monday, Wednesday, and Friday. The sunitinib group was treated daily with 20 mg/Kg sunitinib in a carboxymethylcellulose solution by oral gavage. The combination group was treated with both folate-FITC and sunitinib. Tumor sizes were measured using digital calipers on alternating days, and tumor volumes were calculated using the equation ½ (L×W²). Progressive tumor volume changes for the study groups were graphed in GraphPad Prism, and the results are shown in FIG. 2.

Once it was determined that a study needed to be suspended due to tumor size, all animals in the treatment groups were euthanized within 1-2 days and their tumors and spleens harvested for freezing and analysis by flow cytometry. Mice were euthanized by CO₂ asphyxiation, and their tumors and spleens were removed with the aid of dissecting scissors and tweezers and washed in PBS to remove any excess fur or blood. Both tissues were then weighed in order to obtain a second set of data to corroborate tumor volume measurements and to determine whether tumor cells had metastasized to the spleen.

Folate-FITC is the compound shown in clause 13. The compounds shown in clauses 14 and 15 are equivalent to axitinib (Inlyta®) and sunitinib (Sutent®), respectively. Folate-FITC can be synthesized as described by Kennedy, M. D., et al. in Pharmaceutical Research, Vol. 20(5); 2003.

### Example 2

### Sunitinib/Folate-FITC Combination Therapy in L1210A Tumors

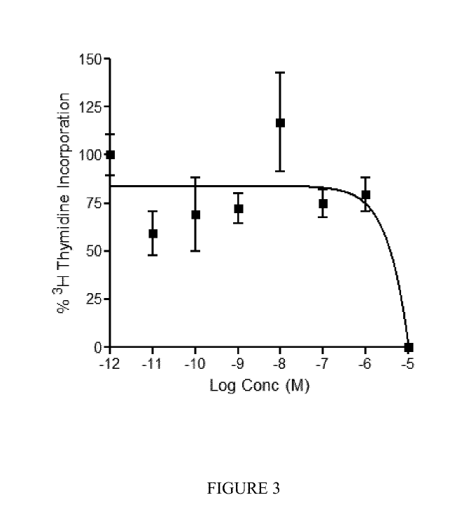

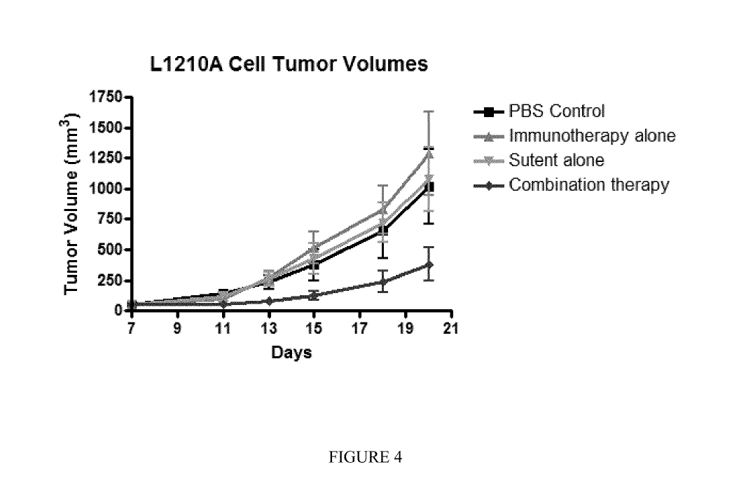

The effect of the combination therapy strategy of Example 1 was evaluated on an alternative tumor type. The L1010A lymphocytic leukemia cell line was selected due to its high FR expression levels and its ability to grow in immune competent mice. Female DBA/2 mice (purchased from Harlan Laboratories in Indianapolis, Ind.) were used in this model. Mice were injected subcutaneously with 1×10⁶ cells suspended in PBS on the left flank. The tumors were palpable by day 5 and had reached 50 mm³ by day 7 at which point therapy was initiated. Originally, a pilot study was conducted to ensure that sunitinib alone had therapeutic efficacy in this particular tumor type, as shown in FIG. 3. Having obtained confirmation of the cell line's response to sunitinib, a full therapeutic study including PBS control, folate-FITC alone, sunitinib alone, and combination groups was conducted with 5 mice in each group using the same doses of sunitinib and treatment schedules described for the Renca study in Example 1. Doses of 500 nmols/Kg of folate-FITC in phosphate buffered saline (PBS) by intraperitoneal injection were used in the folate-FITC alone treatment and the combination treatment. A graph summarizing the tumor volumes for each group over the treatment period is shown in FIG. 4.

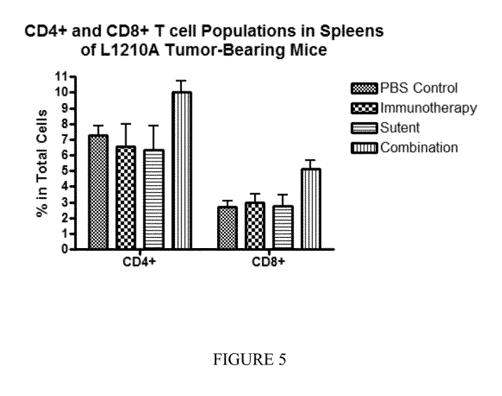

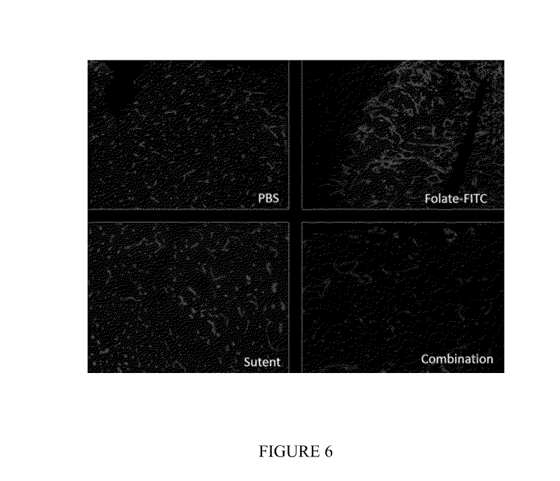

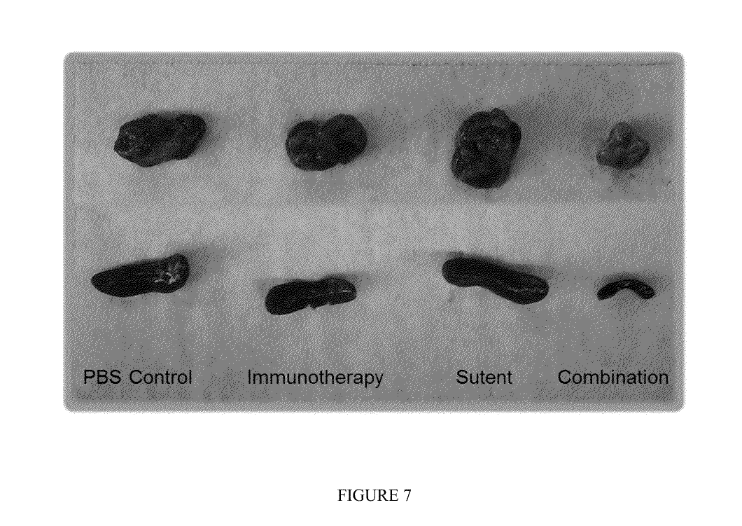

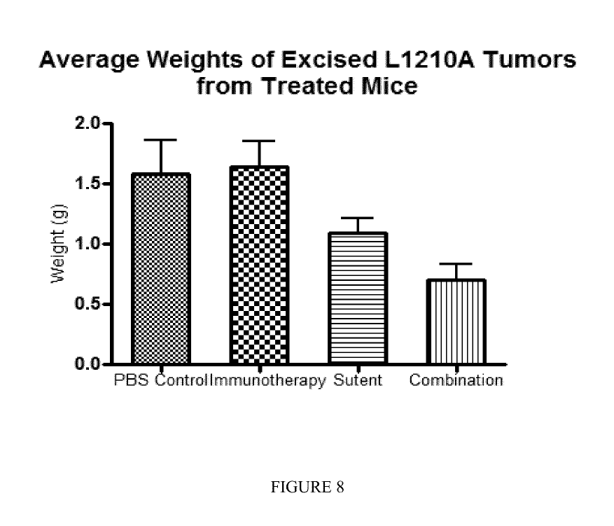

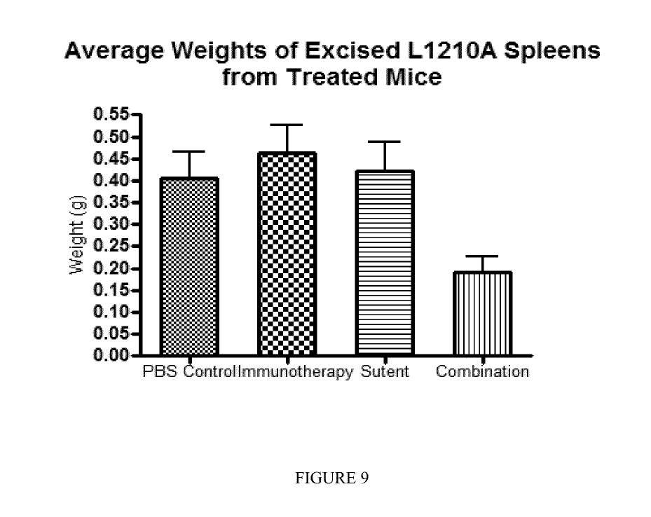

All treatment groups were terminated when tumors in the PBS control group reached 1500 mm³ and tumors and spleens were harvested for analysis. Images of excised tumors and spleens were taken, as shown in FIG. 7. The excised tumors were digested using a collagenase cocktail and single cell suspensions were stained for the presence of FR, macrophages, MDSC, and T cells and analyzed by flow cytometry. Average weights of excised L1210A tumors from treated mice are shown in FIG. 8. The intra-tumoral vasculature content in mice treated with PBS, folate-FITC alone, sunitinib alone, or a combination of folate-FITC and sunitinib was imaged, as shown in FIG. 6. The combination of folate-FITC with sunitinib combination therapy dramatically decreased intra-tumoral vasculature content in comparison to mice treated with PBS or either treatment alone. Portions of each tumor were also cryopreserved for sectioning and immunohistochemistry staining. Average weights of spleens of L1210A tumor-bearing mice are shown in FIG. 9. Harvested spleens were also processed into single cell suspensions and analyzed for immune cell content. The percentage of CD4+ and CD8+ cytotoxic T cell in spleens of L1210A tumor-bearing mice treated with PBS, folate-FITC alone, sunitinib alone, or a combination of folate-FITC and sunitinib was determined, as shown in FIG. 5. The combination of folate-FITC with sunitinib increased both CD4+ and CD8+ cytotoxic T cell levels in L1210A tumor-bearing mice.

### Example 3

### Sunitinib/Folate-FITC Combination Therapy in the M109 Lung Cancer Model

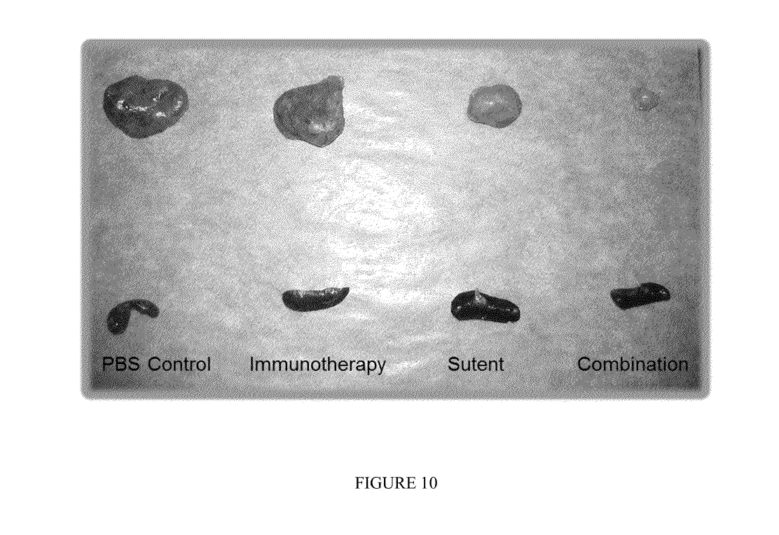

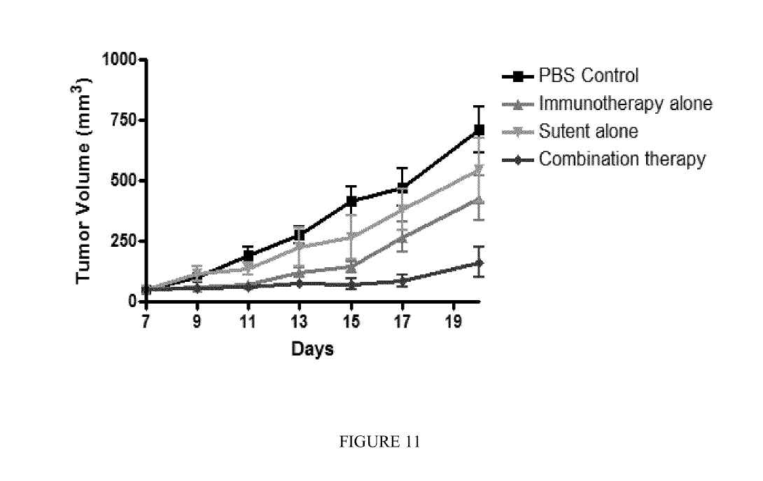

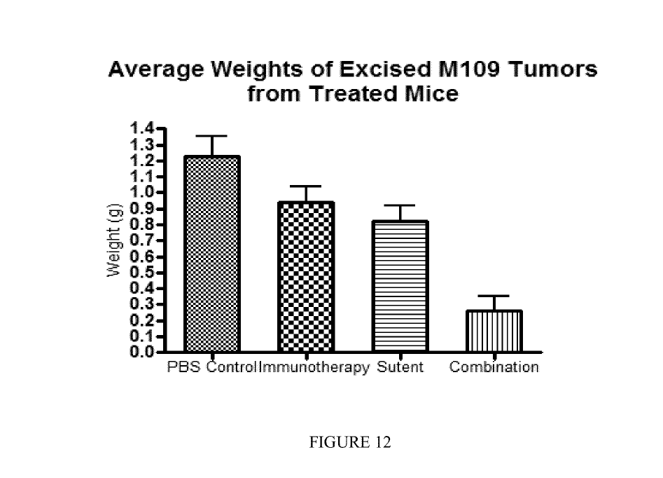

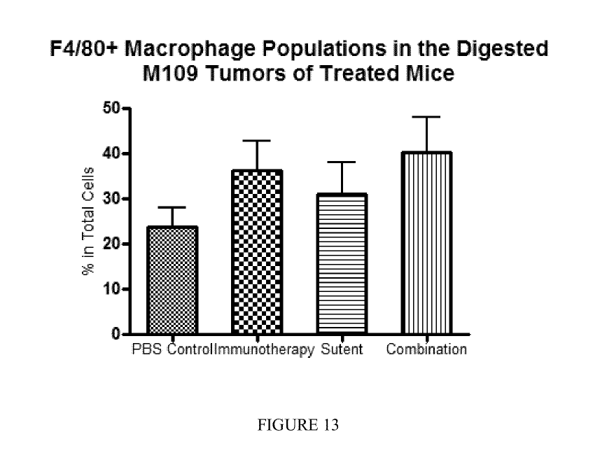

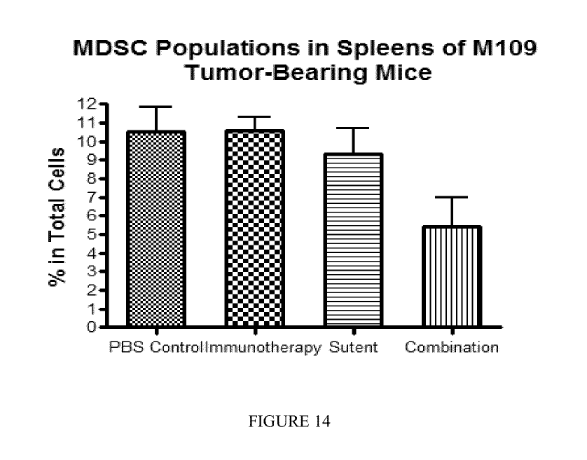

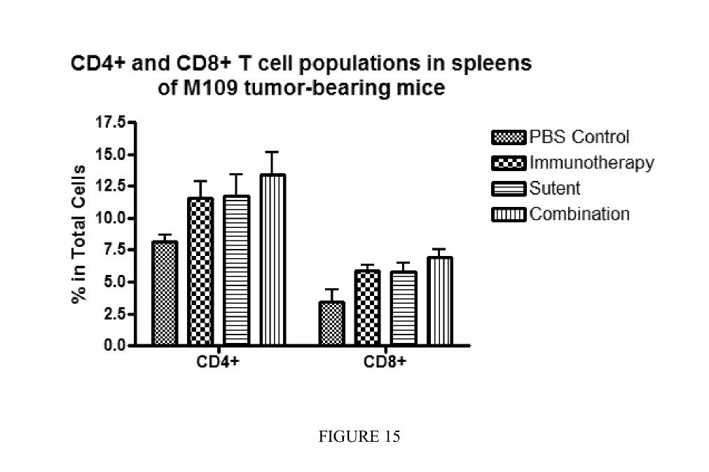

The same therapeutic approach in Example 2 was evaluated in a second FR+ tumor model to confirm that the therapy is a candidate for treating human cancers that overexpress FR. Solid M109 lung tumors were established on the shoulders of KLH-FITC immunized Balb/c mice (5-6 weeks old from Harlan Laboratories, Indianapolis, Ind.) by injecting them subcutaneously with 1×10⁶ of syngeneic M109 cells suspended in media containing 1% female Balb/c serum. The mice were treated according to the same dosing schedule described in Examples 1 and 2. The groups of mice were treated with PBS, folate-FITC alone (500 nmols/Kg of folate-FITC in phosphate buffered saline), sunitinib alone, or a combination of folate-FITC (500 nmols/Kg of folate-FITC in phosphate buffered saline) and sunitinib. Following completion of the study and termination of treatment, the treated animals were sacrificed and tumors and spleens were harvested for analysis as described in Example 2. Images of excised tumors and spleens were taken, as shown in FIG. 10. A plot summarizing the tumor volumes for each treatment group (n=5) for the duration of the study is shown in FIG. 11. Average weights of tumors of M109 tumor-bearing mice are shown in FIG. 12. Bar graphs detailing the different levels of macrophages, MDSCs, and both CD4+ and CD8+ T cells in the analyzed tumors and spleens of the different treatment groups are shown in FIGS. 13-15.

### Example 4

### Axitinib/Folate-FITC Combination Therapy in L1210A Lymphoma Tumors

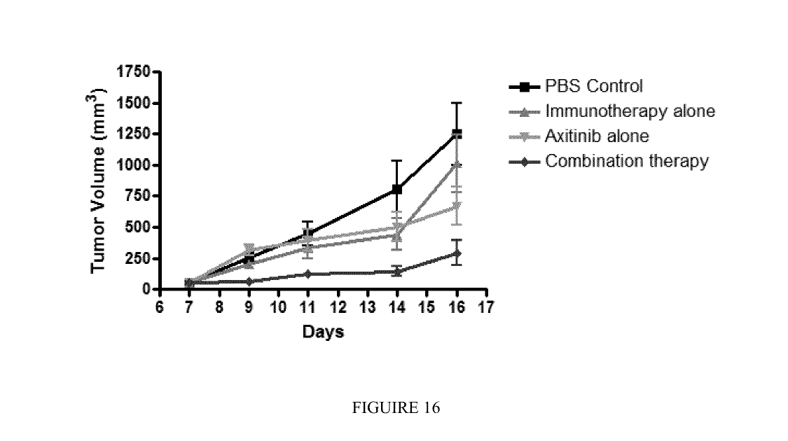

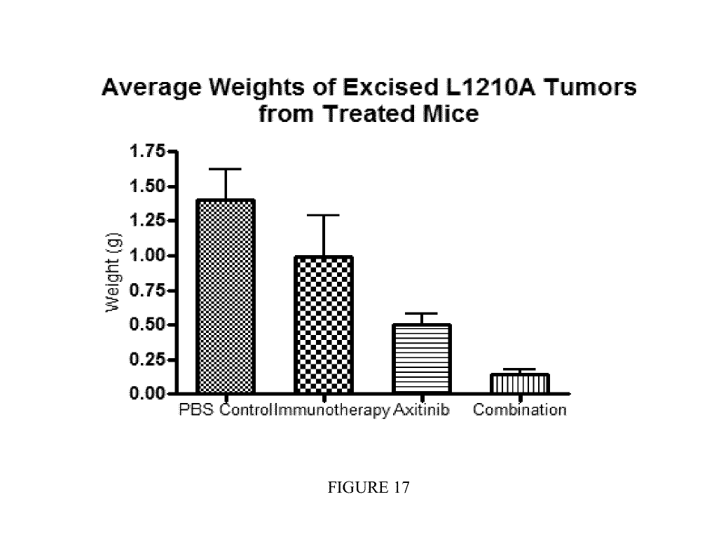

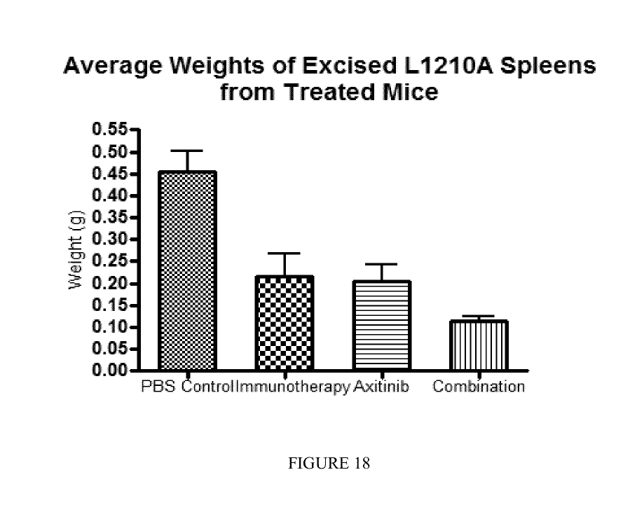

A second RTK inhibitor, axitinib, was studied in combination with folate-FITC on one of the tumor models. The combination therapy was tested in the L1210A lymphoma model following the experimental methods detailed in Example 2, using an axitinib concentration of 15 mg/Kg administered daily by oral gavage in place of sunitinib. The groups of mice were treated with PBS, folate-FITC alone (500 nmols/Kg of folate-FITC in phosphate buffered saline), axitinib alone, or a combination of folate-FITC and axitinib. A plot summarizing the tumor volumes for each treatment group for the duration of the study is shown in FIG. 16. Average weights of tumors and spleens of L1210A tumor-bearing mice are shown in FIGS. 17 and 18, respectively.

What is claimed is:

1. A method of treating a cancer in a patient in need of such a treatment, the method comprising the steps of
administering to the patient a therapeutically effective amount of an immunogen conjugate,
administering to the patient a therapeutically effective amount of a folate conjugate comprising a folate linked to a targeting hapten, and
administering to the patient a therapeutically effective amount of a tyrosine kinase inhibitor.

2. The method of claim 1 wherein the cancer is a folate receptor expressing cancer.

3. The method of claim 2, wherein the cancer is selected from the group consisting of a carcinoma, a sarcoma, a lymphoma, a melanoma, a mesothelioma, a leukemia, an adenocarcinoma, and a myeloma.

4. The method of claim 3, wherein the cancer is selected from the group consisting of lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, oral cancer, laryngeal cancer, testicular cancer, liver cancer, non-small cell lung cancer, cancer of the adrenal gland, cancer of the urethra, prostate cancer, chronic leukemia, acute leukemia, lymphocytic lymphoma, pleural mesothelioma, nasopharyngeal carcinoma, cancer of the bladder, Burkitt's lymphoma, cancer of the ureter, kidney cancer, renal cell carcinoma, brain cancer, and pituitary adenoma.

5. The method of claim 1, wherein the immunogen conjugate comprises a carrier linked to an antigenic hapten.

6. The method of claim 1, wherein the immunogen conjugate has the formula 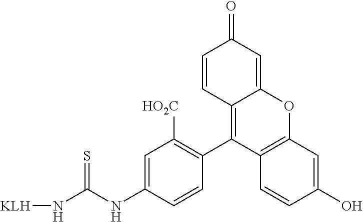
or a pharmaceutically acceptable salt thereof.

7. The method of claim 1, wherein the folate has the formula 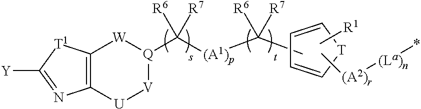
wherein X and Y are each-independently selected from the group consisting of halo, R², OR³, SR³, and NR⁴R⁵;
U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, NR^{8a}, and —(R⁸)C═C(R⁸)—; T¹ is —N═C(X)— or —NH—C(O)—;
A¹ and A² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;
R¹ is selected from the group consisting of hydrogen, halo, C₁-C₁₂ alkyl, and C₁-C₁₂ alkoxy; R², R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;
R³ is in each instance independently selected from the group consisting of hydrogen, C₁-C₁₂ alkyl, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;
R⁴, R^{4a}, R^{4b}, R⁵, and R^{5b} are each independently selected from the group consisting of hydrogen, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;
R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;
R^{8a} is hydrogen, C₁-C₁₂ alkyl, and C₁-C₁₂ alkoxy, or a bond to (A²)ᵣ-(L^{a})ₙ; R⁸ is independently selected in each instance from R¹ or a bond to (A²)ᵣ-(L^{a})ₙ;
L^{a} is a divalent linker as described herein;
n, p, r, s and t are each independently either 0 or 1; and
* denotes the attachment point to the remainder of the conjugate;
or a pharmaceutically acceptable salt thereof.

8. The method of claim 1, wherein the folate is folate.

9. The method of claim 1, wherein the antigenic hapten and the targeting hapten have the formula 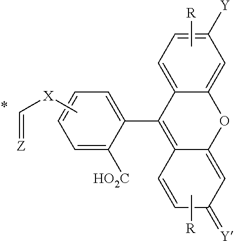
wherein X is O, NH, or S, and where * denotes the point of attachment of the hapten to the rest of the conjugate; Z is O or S; Y is OR^{a}, NR^{a}₂, or NR^{a}₃⁺; and Y′ is O, NR^{a}, or NR^{a}₂⁺, where R^{a} is hydrogen or C₁-C₆ alkyl; and each R independently represents from 0-2 substituents independently selected in each instance from the group consisting of halogen, hydroxyl, C₁-C₆ alkyl, C₁-C₆ alkyloxy, amino, C₁-C₆ alkylamino, (C₁-C₆ alkyl)₂amino, nitro, cyano, cyanate, thiocyanate, C₁-C₆ alkylcarbonyl, C₁-C₆ alkyloxycarbonyl, C₁-C₆ alkylaminocarbonyl, C₁-C₆ alkylcarbonylamino, C₁-C₆ alkylcarbonyloxy, C₁-C₆ alkylthio, SO₃H, and CO₂H;
or a pharmaceutically acceptable salt thereof.

10. The method of claim 6, wherein the folate conjugate has the formula 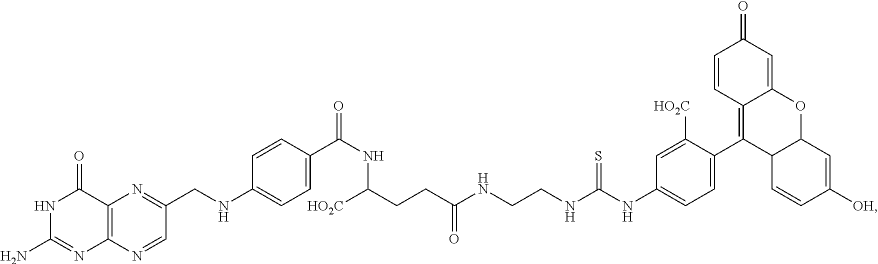 [SMILES: Nc1nc2ncc(CNc3ccc(C(=O)NC(CCC(=O)NCCNC(=S)Nc4ccc(C5=C6C=CC(=O)C=C6OC6C=C(O)C=CC56)c(C(=O)O)c4)C(=O)O)cc3)nc2c(=O)[nH]1]
or a pharmaceutically acceptable salt thereof.

11. The method of claim 10, wherein the tyrosine kinase inhibitor has the formula 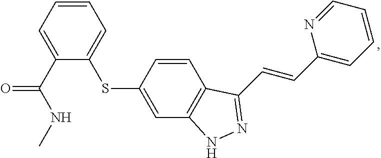 [SMILES: CNC(=O)c1ccccc1Sc1ccc2c(/C=C/c3ccccn3)n[nH]c2c1]
or a pharmaceutically acceptable salt thereof.

12. The method of claim 10, wherein the tyrosine kinase inhibitor has the formula 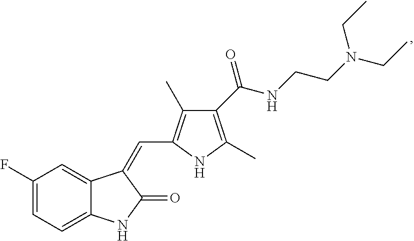 [SMILES: CCN(CC)CCNC(=O)c1c(C)[nH]c(/C=C2\C(=O)Nc3ccc(F)cc32)c1C]
or a pharmaceutically acceptable salt thereof.

14. The method of claim 1, wherein the folate conjugate, the immunogen conjugate, and/or the tyrosine kinase inhibitor are administered in a parenteral dosage form wherein the parenteral dosage form is selected from the group consisting of intradermal, subcutaneous, intramuscular, intraperitoneal, intravenous, and intrathecal.

15. The method of claim 1, wherein the therapeutically effective amount is from about 0.5 mg/m² to about 6.0 mg/m².

16. The method of claim 1, further comprising the step of detecting folate receptor expression by the cancer.

17. The method of claim 16, wherein the detecting is performed by SPECT imaging.

18. The method of claim 16, wherein the step of detecting comprises administering to the patient an imaging conjugate of the formula 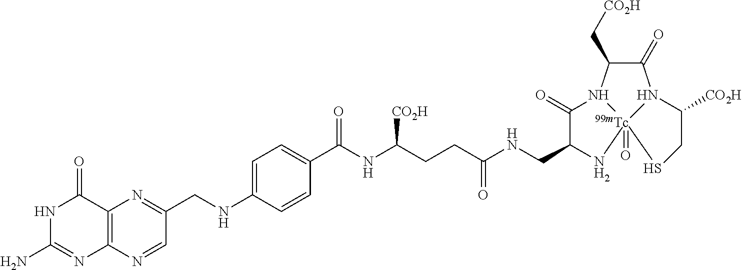
or a pharmaceutically acceptable salt thereof.

19. The method of claim 1, further comprising administering an adjuvant to the patient.

20. The method of claim 19, wherein the adjuvant is GPI-0100.
